(19) United States
(12) Patent Application Publication
(10) Pub. No.: US 2020/0179445 A1
(43) Pub. Date: Jun. 11, 2020

(54) ANTIMICROBIAL COMPOSITION COMPRISING A POLYSACCHARIDE, A STABILIZING AGENT AND TRIIODIDE, METHOD OF PREPARATION THEREOF AND USE THEREOF

(71) Applicant: CONTIPRO A.S., Dolni Dobrouc (CZ)

(72) Inventors: Radovan BUFFA, Humenne (SK); Veronika STEPANKOVA, Chotenov (CZ); Ivana BASARABOVA, Medzilaborce (SK); Josef CHMELAR, Usti nad Orlici, Hylvaty (CZ); Katerina MAIRYCHOVA, Pardubice, Pardubicky (CZ); Vojtech ZAPOTOCKY, Zamberk (CZ); Tomas PITUCHA, Chrudim, Chrudim I (CZ); Katerina KNOTKOVA, Chocen (CZ); Lubos SOBOTKA, Hradec Kralove, Malsovice (CZ); Kristyna LIPENSKA, Letohrad (CZ); Vladimir VELEBNY, Zamberk (CZ)

(21) Appl. No.: 16/619,402

(22) PCT Filed: Jun. 1, 2018
(86) PCT No.: PCT/CZ2018/050028

§ 371 Date: Dec. 4, 2019

(30) Foreign Application Priority Data
Jun. 5, 2017 (CZ) PV 2017-320

(51) Int. Cl. A61K 33/18; A61K 9/70; A61K 9/00; A61K 47/38; A61K 47/36

(52) U.S. Cl. CPC A61K 33/18; A61K 9/7007; A61K 47/36; A61K 47/38; A61K 9/0024

(57) ABSTRACT

A composition having antimicrobial activity is provided. The composition comprises a polysaccharide, a stabilizer, and a triiodide, where the triiodide decomposition to iodide and volatile iodine can be significantly suppressed by the presence of the stabilizer. Methods of preparing and using the composition and devices prepared therewith are also disclosed. As compared to liquid forms comprising triiodide, stabilized solid forms can be used for a much wider range of applications due to their shape stability and a significantly smaller volume and weight of total material.

### CROSS-REFERENCE TO RELATED APPLICATIONS

This application is the National Stage of International Application No. PCT/CZ2018/050028, filed on 1 Jun. 2018, which claims priority to and all advantages of CZ Application No. PV2017-320, filed on 5 Jun. 2017, both of which are incorporated herein by reference in their entirety.

### TECHNICAL FIELD

Antimicrobial composition comprising a polysaccharide, a stabilizing agent and triiodide, method of preparation thereof and use thereof.

### BACKGROUND

Sodium alginate is an anionic polysaccharide with a wide range of biomedical applications. Its main advantage is its biocompatibility and the ability to form gels, therefore it is often used in hydrogels preparation, in the field of wound healing and in tissue engineering (see, e.g. Lee K. Y. and David J. Mooney D. J., Progress in Polymer Science 37, 1, 106-126, 2012).

Carboxymethyl cellulose is an anionic polysaccharide used mainly in the food industry to thicken and stabilize emulsions. For non-food products, it has been used for example in lubricants, paints, laxatives and detergents. This polysaccharide is widely used in the field of wound healing, where the price and interesting mechanical properties are probably the most advantageous (see, e.g. Ramli A., Wong T. W., International Journal of Pharmaceutics, 403, 7, 73-82, 2011).

Oxycellulose is cellulose oxidized in the position 6 of cycle to carboxylic acid. Thus, it is an anionic polysaccharide, which is known especially for its haemostatic effects, and is therefore widely used for a variety of medical and pharmaceutical applications, for example in the field of wound healing, where, in addition to haemostatic properties, biodegradability and sorption properties are of great advantage (see, e.g. Bajerova M. et al., Advances in polymer technology, 28, 199-208, 2009).

Hydroxyethyl cellulose is a cellulose derivative modified on certain OH groups by —CH₂—CH₂—OH group. It is not as well soluble in protic systems as oxycellulose, but due to its gelation properties it is widely used in cosmetics, cleaning solutions and lubricants. For wound healing it is used especially in combination with other polysaccharides, such as gellan gum (see, e.g.

Schmidt R. and Winter G., EP1888134 A2)

Hyaluronic acid is a non-sulphated glycosaminoglycan, comprising of two repeating units of D-glucuronic acid and N-acetyl-D-glucosamine.

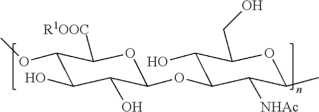

wherein

R¹ is H or Na.

This hydrophilic polysaccharide with a molecular weight in the range from 5×10³ to 1×10⁶ g.mol⁻¹ forms a part of the skin, connective tissues, and synovial joint fluid and plays a significant role in a number of biological processes such as the organization of proteoglycans, cell hydration and differentiation (see, e.g. Balazs E., Structural Chemistry, 20, 341-349, 2009; Aya K. L. and Stern R. Wound Repair and Regeneration 22, 579-593, 2014). Due to the fact that it is naturally in the human body, and thus biodegradable, it is a suitable substrate for tissue engineering or a carrier of biologically active substances (see, e.g. Mortisen D. et al., Biomacromolecules, 11 (5), 1261-1272, 2011; Collins M. N. and Birkinshaw C., Carbohydrate Polymers, 92, 1262-79, 2013). For example, injection application of hyaluronic acid into osteoarthritic joints is well known, where significant improvement in the joint functionality has been observed (see, e.g. Muzzarelli R. A. et al., Review: Carbohydrate Polymers, 89, 723-739, 2012). This polymer is also known to support the wound healing process due to its biological properties (see, e.g. Nyman E. et al., J. Plast. Surg. Hand Surg. 47 (2), 89-92, 2013).

Chemical Modifications of Hyaluronic Acid and its Forms

Many methods of chemical modification of hyaluronic acid in order to change its physical and biological properties are understood in the art (see, e.g. Burdick J. A. and Prestwich G. D. Adv. Mater. 23, 41-56, 2011). In case a substantial change in solubility is desired for a particular application, the most common solution is the covalent bonding of the hydrophobic chain in the form of a biodegradable ester link to the polymer structure (see, e.g. Kettou et al. CZ Application No. PV 2009-399, Buffa et al. WO2010105582). Various forms can be made from such modified materials, for example fibers (see, e.g. Scudlova et al. EP2925916 A1), knitted fabrics and plaited fabrics (see, e.g. Pitucha et al., CZ 306354), self-supporting films (see, e.g. Foglarova et al. CZ Application No. PV2015-166; Foglarova M. et al. Carbohydrate Polymers 2016, 144, 68-75) or nanofibrous layers (see, e.g. Ruzickova J. et al. CZ Application No. PV2013-913). Non-woven fabrics are made up of staple microfibers that are prepared by wet spinning in a non-stationary coagulation bath. The coagulation bath comprises of 100% C₁-C₃ alcohol. The precipitated fibers are then shortened by grinding, filtered to substrate, dried and compressed. In this way, non-woven fabrics can be prepared from HA with molecular weight 60-3,000 kg.mol⁻¹. The resulting layer may remain adhered to the substrate or be separated from the substrate as a self-supporting layer with an area weight greater than 5 g.m⁻².

Hyaluronic Acid and Triiodide

Forms of iodine with an oxidation state higher than −1 (I⁻) are well known as biocompatible antiseptic and disinfectant substances. One of the most widespread forms is triiodide (oxidation grade −⅓), which is subject of reversible decomposition to molecular iodine (12) and iodide (I⁻). The molecular iodine passes into the gaseous state, so the solids containing the triiodide gradually lose their oxidative capabilities due to the sublimation of I₂. For this reason, the triiodide is used especially in the form of solutions. An example is the so-called Lugol solution (i.e., potassium triiodide in water), which, due to its biocompatibility and efficacy, is suitable for a wide range of applications associated with antiseptic or disinfecting action. Its slight disadvantage is that it can cause scarring and also temporary change of the skin color. These deficiencies have been overcome by an addition of hyaluronic acid, which considerably suppresses scarring and generally significantly contributes to the healing process. The document CZ Pat. Pub. No. 12015 discloses a preparation for a bandage adhesion prevention comprising a physiologically acceptable hyaluronic acid salt having a molecular weight of 200,000 to 2,500,000, iodine and potassium iodide. The preparation is in the form of a sterile aqueous solution or gel and is able to make the wound healing faster. This use of a solution of hyaluronic acid and potassium iodide (under the commercial name Hyiodine) for topical wound healing applications has been published in several papers (see, e.g. Bezdekova B. et al. Veterinarstvi 54, 516-519, 2004; Frankova J. et al. Journal of Materials Science: Materials in Medicine 17, 891-898, 2006; Slavkovsky R. et al. Clinical and Experimental Dermatology 35, 4, 373-379, 2010).

In terms of storage, transport and possible other in situ applications, use of triiodide with a polysaccharide in the form of a solution represents significant limitations. The volume of the material (liquid form) is considerably larger than the volume of the analogous solid form and further other possibilities of in situ use are considerably limited due to the solution shape instability (flowing). Additionally, the liquid form is limited by the form of the package where it is very difficult to use other types of packaging materials for longer storage than the standard silicate glass which is fragile, due to the oxidative activity of the triiodide. Attempts to prepare a solid material containing a polysaccharide and triiodide have not been successful due to the instability of the triiodide in the absence of a solvent. The presence of the solvent inhibits the process of the molecular I₂ sublimation and allows re-bonding with I⁻ in the form of the triiodide I₃⁻. Therefore, during evaporation of the solvent, the Lugol solution quickly loses the active ingredient (I₂), which sublimates from the solid material, and in view of the long-term storage of some of the triiodide-containing final forms, it is a crucial problem.

The document CZ Pat. Pub. No. 22394 describes an antimicrobial mixture for wound healing support and wound dressing for healing support with an antimicrobial effect. The mixture comprises a physiologically active hyaluronic acid salt, alternatively other polysaccharides and substances with antimicrobial activity, and further an electrolyte, e.g., potassium iodide. The mixture can be in the form of a chemical or physical mixture, wherein the chemical mixture is preferably an aqueous solution and the physical mixture is preferably a layer of polysaccharide fibers which contain an antimicrobial substance in their structure. The dressing is suitable for healing of surface wound. The disadvantage of this solution is in particular the essential presence of an antimicrobial agent other than triiodide, which involves the risk of local skin irritation, toxicity or allergic reaction.

Accordingly, it is desirable to provide an improved antimicrobial composition and methods relating to the same. Furthermore, other desirable features and characteristics will become apparent from the subsequent summary and detailed description and the appended claims, taken in conjunction with the foregoing technical field and background.

### BRIEF SUMMARY

As contemplated herein, the preparation of solid forms comprising a polysaccharide, triiodide and a stabilizer, which is believed to significantly slow down the sublimation of the active iodine from the solid material, is provided.

An antimicrobial composition is provided. The composition comprises a polysaccharide or chemically modified derivative thereof. The polysaccharide or the chemically modified derivative thereof has a molecular weight in a range from 5×10³ to 1×10⁶ g.mol⁻¹. The polysaccharide or the chemically modified derivative thereof is selected from the group of hyaluronic acid, sodium alginate, oxycellulose, carboxymethyl cellulose, hydroxyethyl cellulose, modified hyaluronic acid, and combinations thereof. One or more of the -OH groups of the modified hyaluronic acid are substituted by a —O—CO—R² group or one or more of the —CO—OH groups of the modified hyaluronic acid are substituted by a —CO—OR² group. R² is a linear or aromatic chain with carbon atoms content C₁-C₁₅. The composition further comprises a thiazole-based stabilizer selected from the group of thiamine, oxythiamine hydrochloride, 5-(2-hydroxyethyl)-3,4-dimethyl thiazolium iodide, 3-benzyl-5-(2-hydroxyethyl)-4-methyl thiazolium bromide, and combinations thereof. The composition further comprises sodium or potassium triiodide.

The subject matter as described herein are formulations comprising a polysaccharide and/or a chemically modified derivative thereof or a mixture of polysaccharides and/or derivatives thereof, sodium or potassium triiodide and a stabilizer of the general formula X,

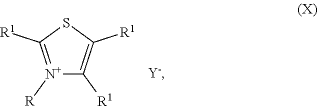

wherein
R is -alkyl, aromatic, heteroaromatic, linear or branched chain C₁-C₃₀, optionally containing N or O atoms,
R¹ is -alkyl, aromatic, heteroaromatic, linear or branched chain C₁-C₃₀, optionally containing N or O atoms, or —H, where R¹ in the compound of the formula X are independently the same or different,
and Y is a chloride, bromide or iodide anion.

In some embodiments, the final materials are prepared as various solid forms such as self-supporting films, lyophilizate, staple fiber layer (non-woven fabric), endless fiber, woven fabric, knitted fabric, plaited fabric or nanofibers layer.

The polysaccharide or chemically modified derivative thereof, has a molecular weight in the range from 5×10³ to 1×10⁶ g.mol⁻¹, and the source of the triiodide anion is potassium iodide or sodium iodide and molecular iodine I₂.

The polysaccharide comprises, or is selected from the group of, for example, hyaluronic acid, sodium alginate, oxycellulose, carboxymethyl cellulose, hydroxyethyl cellulose or a chemically modified hyaluronic acid derivative which has some —OH groups replaced by —O—CO—R² group and/or —CO—OH groups replaced by —CO—OR² group, where R² is a linear or aromatic chain containing carbon atoms C₁-C₁₅, or mixtures of various polysaccharides and/or polysaccharide derivatives with an optional ratio of individual components. In addition, in various embodiments, the composition or the final medical device may contain other substances, including, but not limited to, polyethylene oxide, acetic acid etc.

A method of preparation is also provided, where two approaches of the stabilized triiodide introduction can be used.

Procedure 1—Coating:

In certain embodiments, one approach is to prepare a solution of a stabilizer of the general formula (X) (i.e., “stabilizer X”) and sodium or potassium triiodide in an ethanol/water solvent mixture, and to apply this solution to the finished form of the medical device, which is based on a polysaccharide or a derivative thereof and/or a mixture of polysaccharides and/or derivatives thereof The application time may be in the range from 10 minutes to 72 hours at a temperature in the range from 5 to 40° C. In some embodiments, the solution can be applied on the medical device either by spraying or by immersing the medical device into the solution, optionally for 5 to 15 hours. More specifically, the process may be carried out by application of 0.2 to 10% (w/w) solution of triiodide and stabilizer X in a molar ratio of 1/1 to 1/5, optionally 1/1, in a solvent mixture of ethanol/water in volume ratio of 3/1 to 9/1, on the surface of the finished final forms of the polysaccharide or derivative thereof or mixture of polysaccharides, optionally either by spraying the solution of the triiodide and the stabilizer or by immersing the final form of a polysaccharide or a derivative thereof or a mixture of polysaccharides and/or derivatives thereof in the solution of the triiodide and stabilizer.

Procedure 2:

In other embodiments, in another approach a mixture comprising a system of a polysaccharide and/or a polysaccharide derivative and/or a mixture thereof, potassium or sodium triiodide and a stabilizer of the general formula X is prepared, whereupon the final form of the composition is formed. More specifically, the triiodide at a concentration of 0.2 to 10% (based on the total weight of all polysaccharides and/or derivatives thereof) and stabilizer X in a molar ratio of triiodide/stabilizer in the range from 1/1 to 1/5, optionally 1/1.1, may be added to a 0.2 to 6% (w/w) solution of a polysaccharide or a derivative thereof or mixture of polysaccharides and/or derivatives thereof in water and acetic acid in a volume ratio of 20/1 to 200/1, optionally 100/1. After a thorough homogenization and eventually after an addition of other substances, the final form of the composition may be formed.

In this and other embodiments, by applying the Procedure 2 for example, a material is formed wherein the triiodide anion with the stabilizer are more homogeneously distributed throughout the bulk of the material. This procedure can be used, for example, to prepare the material in the form of a lyophilizate.

In this and other embodiments, by applying the Procedure 1 for example, a material is formed wherein the triiodide anion with the stabilizer are mainly on or near the surface of the respective form. This process can be used for a variety of forms: self-supporting films, lyophilizate, staple fiber layer (non-woven fabric), endless fiber, woven fabric, knitted fabric, plaited fabric or nanofiber layer.

The following chemical compounds can be used as stabilizers of the general formula X: Thiamine (B1), oxythiamine hydrochloride (OB1), 5-(2-hydroxyethyl)-3,4-dimethylthiazolium iodide (TH), a 3-benzyl-5-(2-hydroxyethyl)-4-methylthiazolium bromide (BTH) and combinations thereof.

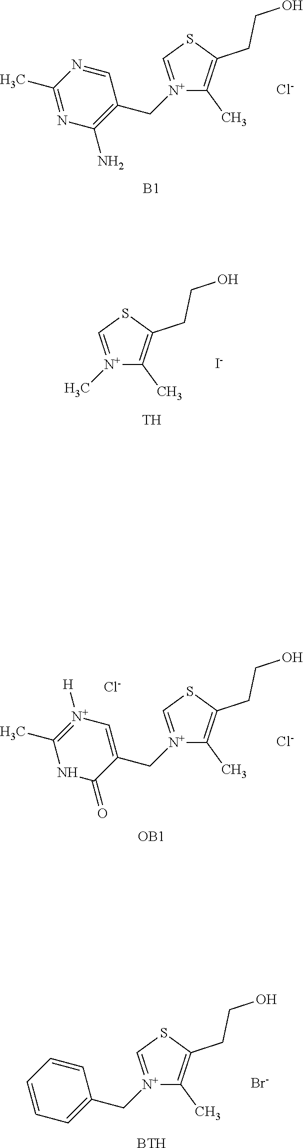

In various embodiments, the effectiveness of the stabilizers was clearly demonstrated when trying to prepare lyophilizates containing Iₛ⁻ in the absence of thiazole salts. The active iodine content after lyophilization (high vacuum) was 100 times lower than that of the analogous lyophilizates containing the stabilizer.

In some embodiments, the invention also relates to a medical device which comprises an antimicrobial composition as defined above and is in the form of a wound dressing or an implantable medical device.

### BRIEF DESCRIPTION OF THE DRAWINGS

Other advantages of the disclosed subject matter will be readily appreciated, as the same becomes better understood by reference to the following detailed description when considered in connection with the accompanying drawings wherein:

FIG. 1 is an image illustrating use of a non-limiting embodiment of an antimicrobial composition;

FIG. 2 is another image illustrating use of a non-limiting embodiment of the antimicrobial composition; and

FIG. 3 is another image illustrating use of a non-limiting embodiment of the antimicrobial composition.

### DETAILED DESCRIPTION

An antimicrobial composition comprising a polysaccharide or a derivative thereof or a mixture of polysaccharides and/or derivatives thereof, a stabilizing agent and sodium or potassium triiodide is provided. In various embodiments, the composition results in the stabilization of various types of solid forms containing the polysaccharide and/or its chemically modified derivative and/or their mixture and the iodine in the form of a triiodide anion (I₃⁻). As stabilizing agents, or stabilizers, significantly suppressing the decomposition of the triiodide anion to iodine (I₂) and iodide (I⁻), the cationic heterocyclic compounds of general formula X may be successfully used,

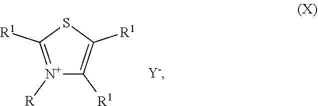

wherein
R is -alkyl, aromatic, heteroaromatic, linear or branched chain C₁-C₃₀, optionally containing N or O atoms,
R¹ is -alkyl, aromatic, heteroaromatic, linear or branched chain C₁-C₃₀, optionally containing N or O atoms, or —H, where R¹ in the compound of the formula X are independently the same or different, and
Y is a chloride, bromide or iodide anion.

A method of preparation is also provide. The method may be to prepare solid forms. Two procedures can be used. However, the method of preparation is not limited to these two procedures. Procedure 1: The triiodide and the stabilizer are sorbed on the surface of the finished final form. Procedure 2: The triiodide and the stabilizer are added to the system before producing the final form.

In certain embodiments, the difference between Procedures 1 and 2 is that in the Procedure 2 the triiodide anion with the stabilizer are distributed in the bulk of material more homogeneously, whereas in the Procedure 1 the triiodide anion with the stabilizer are preferentially distributed on the surface of the respective form.

As used herein, the term “form” refers to types of materials, such as, for example, thin film, lyophilizate, staple fiber layer, endless fiber, woven fabric, plaited fabric or nanofibrous layer.

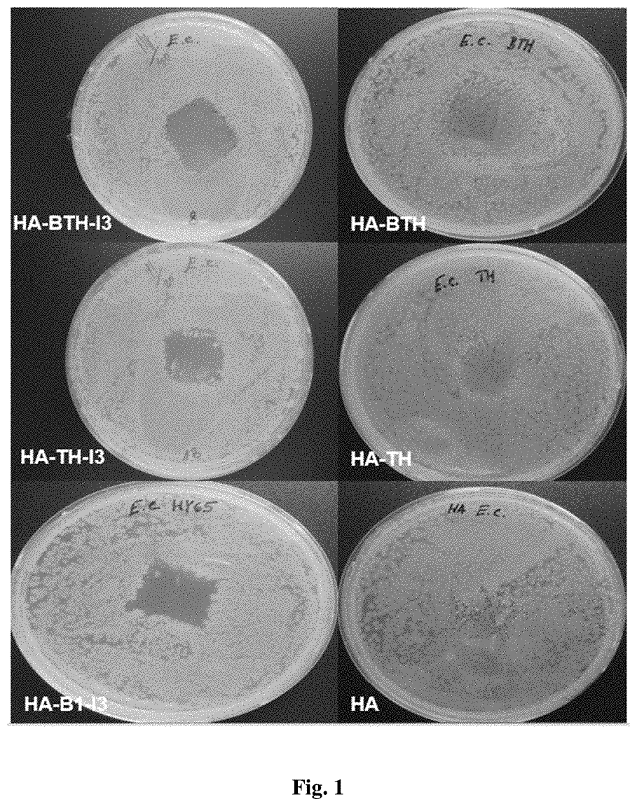

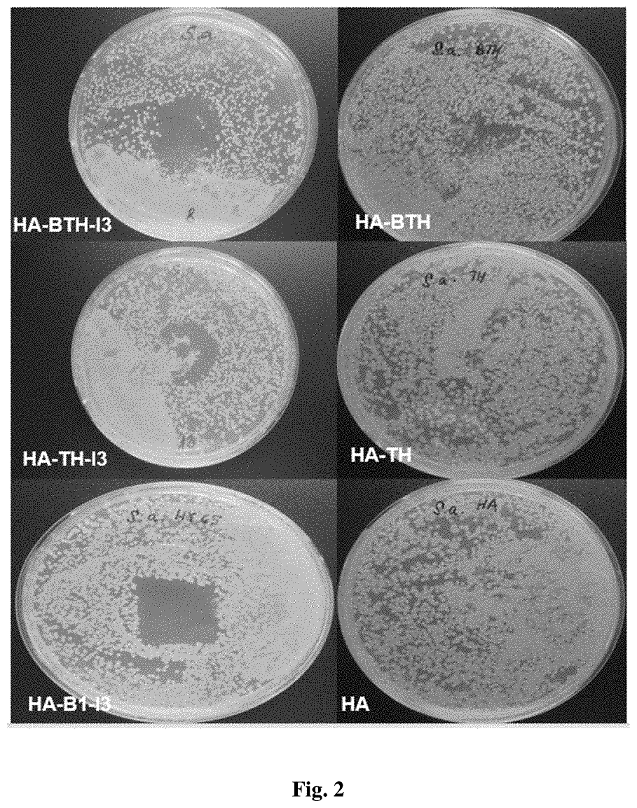

Furthermore, the invention relates to the applications of the prepared solid forms in the fields where a biocompatible and biodegradable material with an antiseptic effect is required. These areas include, but are not limited to, wound dressings or implantable medical devices. FIGS. 1 and 2 provide non-limiting and exemplary comparisons of antimicrobial activity of hyaluronic acid (HA) based lyophilizates prepared by Procedure 2 (the triiodide and the stabilizer are distributed more homogeneously). Triiodide-free materials (HA-TH, HA-BTH, HA-B1, a HA) were tested as controls, exhibiting no inhibitory activity. Materials with the antimicrobial triiodide (HA-TH-I₃, HA-BTH-I₃, a HA-B1-I₃) inhibited the growth of microorganisms. All materials were tested for Escherichia coli (FIG. 1) and Staphylococcus aureus (FIG. 2) strains.

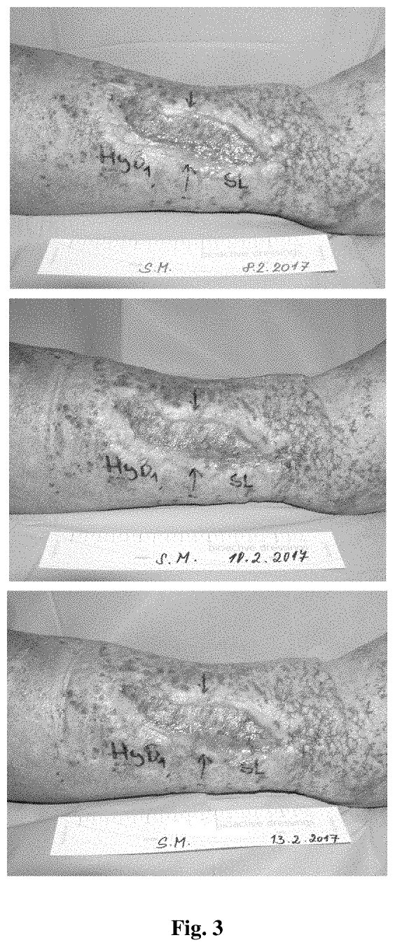

FIG. 3 provides another non-limiting and exemplary comparison of wound healing effect of the HA-vitamin B1-triiodide lyophilizate prepared in Example 13 (in the figure the portion of the wound healed by this preparation is indicated as HyB₁) and of an antimicrobial octenidine-containing lyophilizate based on hyaluronan (in the figure is indicated as SL) at 0, 2 and 5 days in a patient with an open wound on a leg (process described in Example 42). The figure shows a comparable efficacy of both materials.

### EXAMPLES

DS=degree of polysaccharide substitution=100%*(molar amount of a modified polysaccharide unit)/(molar amount of polysaccharide repeating units), as determined by NMR (see, e.g. Kettou et al. CZ Application No. PV 2009-399).

The term equivalent (equiv) used herein refers to the repeating unit of the respective polysaccharide, unless otherwise indicated.

Percentages are reported as percentage by weight, unless otherwise indicated.

The amount of active iodine in %—means an equivalent of oxidation activity rate of the material, which is equivalent to the oxidation activity of the material with the corresponding weight percentage of I₂. Determined by standard redox titration with sodium thiosulphate.

The molecular weight of polysaccharides is weight average molecular weight determined by SEC-MALLS method.

### Example 1

Preparation of Hyaluronan Ethyl Ester

To a solution of hyaluronan (1 g, 300 kg.mol⁻¹) in 40 mL of water, NaOH was added until pH =9. Then 20 mL of dimethyl sulfoxide and 0.08 mL of ethyl iodide were added and the mixture was stirred for 3 days at 45° C. Subsequently, the resulting mixture was precipitated by 140 mL of 100% isopropanol, the solids were filtered off, washed by isopropanol and dried under vacuum. The product (897 mg) was analyzed by NMR.

DS of ester was 6% (determined by NMR).

### Example 2

Preparation of Hyaluronan Benzyl Ester

To a solution of hyaluronan (1 g, 300 kg.mol⁻¹) in 40 mL of water, NaOH was added until pH =9. Then 20 mL of dimethyl sulfoxide and 0.08 mL of benzyl bromide were added and the mixture was stirred for 4 days at 20° C. Subsequently, the resulting mixture was precipitated by 140 mL of 100% isopropanol, the solids were filtered off, washed by isopropanol and dried under vacuum. The product (920 mg) was analyzed by NMR.

DS of ester was 3% (determined by NMR).

### Example 3

Preparation of Lauroyl Hyaluronan

To a solution of hyaluronan (5 g, 250 kg.mol⁻¹) in 100 mL of distilled water, 70 mL of tetrahydrofurane, 4 equivalents of triethylamine and 0.1 equivalents of 4-dimetylaminopyridine were added. Concurrently, lauric acid (4 equivalents) was dissolved in 30 mL of tetrahydrofurane and 7 mL of triethylamine and to this solution 4.8 mL of ethyl-chloroformiate was added at 0-5° C. in 15 minutes. The suspension formed was filtered into the hyaluronan solution and the reaction was stirred for 5 hours at 20° C. Subsequently, the resulting solution was precipitated by an addition of 400 mL of 100% isopropanol, washed with 80% isopropanol, then with 100% isopropanol. The precipitate was dried at 40° C. for 2 days. The degree of substitution was determined by NMR to be 37%.

### Example 4

Preparation of Palmitoyl Hyaluronan

To a solution of hyaluronan (10 g, 250 kg.mol⁻¹) in 300 mL of distilled water, 300 mL of tetrahydrofurane was added. Subsequently 2.5 equivalents of triethylamine, 0.04 equivalents of 4-dimethylaminopyridine and 2 equivalents of palmitic acid anhydride were added to this solution. The resulting solution was stirred at laboratory temperature for 3 hours, then was precipitated by 1 L of 100% isopropanol, washed with 80% isopropanol and dried at 40° C. for 2 days. The degree of substitution was 30% (determined by NMR).

### Example 5

Preparation of Thiamine-KI₃ Solution in Ethanol/Water 3/1

150 mg of I₂ and 225 mg of KI were dissolved in 21 mL of ethanol. 210 mg of thiamine hydrochloride were dissolved in 7 mL of distilled water. Both solutions were mixed at 20° C. and stored at 0-5° C.

### Example 6

Preparation of Thiamine-KI₃ Solution in Ethanol/Water 6/1

150 mg of I₂ and 225 mg of KI were dissolved in 25.7 mL of ethanol. In 4.3 mL of distilled water, 210 mg of thiamine hydrochloride were dissolved. Both solutions were mixed at 20° C. and stored at 0-5° C.

### Example 7

Preparation of Thiamine-KI₃ Solution in Ethanol/Water 9/1

150 mg of I₂ and 225 mg of KI were dissolved in 27 mL of ethanol. 210 mg of thiamine hydrochloride were dissolved in 3 mL of distilled water. Both solutions were mixed at 20° C. and stored at 0-5° C.

### Example 8

Preparation of Thiamine-NaI₃ Solution in Ethanol/Water 3/1

150 mg of I₂ and 203 mg of NaI were dissolved in 21 mL of ethanol. 210 mg of thiamine hydrochloride were dissolved in 7 mL of distilled water. Both solutions were mixed at 20° C. and stored at 0-5° C.

### Example 9

Preparation of Thiamine-NaI₃ Solution in Ethanol/Water 6/1

150 mg of I₂ and 203 mg of NaI were dissolved in 25.7 mL of ethanol. 210 mg of thiamine hydrochloride were dissolved in 4.3 mL of distilled water. Both solutions were mixed at 20° C. and stored at 0-5° C.

### Example 10

Preparation of Thiamine-NaI₃ Solution in Ethanol/Water 9/1

150 mg of I₂ and 203 mg of NaI were dissolved in 27 mL of ethanol. 210 mg of thiamine hydrochloride were dissolved in 3 mL of distilled water. Both solutions were mixed at 20° C. and stored at 0-5° C.

### Example 11

Preparation of Hyaluronan Ethyl Ester-Thiamine-I₃ (HA-B1-I₃) Lyophilizate

40 mg of KI and 27 mg of I₂ were added to a solution of the hyaluronan derivative prepared according to the Example 1 (0.4 g) in 100 mL of distilled water and 0.5 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 4.2%.

### Example 12

Preparation of Hyaluronan Benzyl Ester-Thiamine-I₃ (HA-B1-I₃) Lyophilizate

40 mg of KI and 27 mg of I₂ were added to a solution of the hyaluronan derivate prepared according to the Example 2 (0.4 g) in 100 mL of distilled water and 5 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 4%.

### Example 13

Preparation of Hyaluronan-Thiamine-I₃ (HA-B1-I₃) Lyophilizate

40 mg of KI and 27 mg of I₂ were added to a solution of hyaluronan (0.4 g, Mw 500 kg.mol⁻¹) in 100 mL of distilled water and 1 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 4%.

### Example 14

Preparation of Hyaluronan-Thiamine-I₃ (HA-B1-I₃) Lyophilizate

40 mg of KI and 27 mg of I₂ were added to a solution of hyaluronan (0.4 g, Mw 500 kg.mol⁻¹) in 200 mL of distilled water and 2 mL of acetic acid, and the resulting mixture was stirred for 24 hours at laboratory temperature. A solution of 179 mg of thiamine hydrochloride in 3 mL of distilled water was then added, the resulting solution was homogenized, immediately frozen at −50° C. and lyophilized. The amount of the active iodine was determined by reductive titration with sodium thiosulphate to be 3.5%.

### Example 15

Preparation of Hyaluronan-Thiamine-I₃ (HA-B1-I₃) Lyophilizate

40 mg of KI and 27 mg of I₂ were added to a solution of hyaluronan (0.4 g, Mw 500 kg.mol⁻¹) in 100 mL of distilled water and 1 mL of acetic acid, and the resulting mixture was stirred for 24 hours at laboratory temperature. A solution of 36 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 3.5%.

### Example 16

Preparation of Haluronan-Thiamine-I₃ (HA-B1-I₃) Lyophilizate

40 mg of KI and 27 mg of I₂ were added to a solution of hyaluronan (0.4 g, Mw 80 kg.mol⁻¹) in 20 mL of distilled water and 0.1 mL of acetic acid, and the resulting mixture was stirred for 24 hours at laboratory temperature. A solution of 36 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 2%.

### Example 17

Preparation of Hyaluronan-Thiamine-I₃ (HA-B1-I₃) Lyophilizate—Coating

Hyaluronan in the form of lyophilizate was completely immersed in a solution of NaI₃ in ethanol/water 3/1 (Example 8) for 24 hours at 20° C. Then the lyophilizate was immersed in isopropanol for 2 seconds, pulled out and dried by applying the filter paper from both sides of the material. The amount of the active iodine was determined by reductive titration with sodium thiosulphate to be 1.5%.

### Example 18

Preparation of Hyaluronan-Thiamine-I₃ (HA-B1-I₃) Lyophilizate—Coating

Hyaluronan in the form of lyophilizate was completely immersed in a solution of NaI₃ in ethanol/water 9/1 (Example 10) for 24 hours at 40° C. Then the lyophilizate was immersed in isopropanol for 2 seconds, pulled out and dried by applying the filter paper from both sides of the material. The amount of the active iodine was determined by reductive titration with sodium thiosulphate to be 2%.

### Example 19

Preparation of Hyaluronan-Thiamine-I₃ (HA-B1-I₃) Lyophilizate—Coating

Hyaluronan in the form of lyophilizate was completely immersed in a solution of Kb in ethanol/water 6/1 (Example 6) for 10 minutes at 40° C. Then the lyophilizate was immersed in isopropanol for 2 seconds, pulled out and dried by applying the filter paper from both sides of the material. The amount of the active iodine was determined by reductive titration with sodium thiosulphate to be 1.5%.

### Example 20

Preparation of Hyaluronan-Thiamine-I₃ (HA-B1-I₃) Lyophilizate—Coating

Hyaluronan in the form of lyophilizate was completely immersed in a solution of Kb in ethanol/water 9/1 (Example 7) for 48 hours at 5° C. Then the lyophilizate was immersed in isopropanol for 2 seconds, pulled out and dried by applying the filter paper from both sides of the material. The amount of the active iodine was determined by reductive titration with sodium thiosulphate to be 2%.

### Example 21

Preparation of Hyaluronan-Thiamine-I₃ (HA-B1-I₃) Lyophilizate—Coating

Hyaluronan in the form of lyophilizate was completely immersed in a solution of KI₃ in ethanol/water 3/1 (Example 5) for 10 hours at 20° C. Then the lyophilizate was immersed in isopropanol for 2 seconds, pulled out and dried by applying the filter paper from both sides of the material. The amount of the active iodine was determined by reductive titration with sodium thiosulphate to be 1%.

### Example 22

Preparation of Hyaluronan-Thiazolium Iodide-I₃ (HA-TH-I₃) Lyophilizate

40 mg of KI and 27 mg of I₂ were added to a solution of hyaluronan (0.4 g, Mw 500 kg.mol⁻¹) in 100 mL of distilled water and 1 mL of acetic acid, and the resulting mixture was stirred for 24 hours at laboratory temperature. A solution of 35 mg of 5-(2-hydroxyethyl)-3,4-dimethyl thiazolium iodide in 1 mL of distilled water was then added, the resulting solution was homogenized, immediately frozen at −50° C. and lyophilized. The amount of the active iodine was determined by reductive titration with sodium thiosulphate to be 3%.

### Example 23

Preparation of Hyaluronan-Benzyl Thiazolium Bromide-I₃ (HA-BTH-I₃) Lyophilizate

40 mg of KI and 27 mg of I₂ were added to a solution of hyaluronan (0.4 g, Mw 500 kg.mol⁻¹) in 100 mL of distilled water and 1 mL of acetic acid, and the resulting mixture was stirred for 24 hours at laboratory temperature. A solution of 37 mg of 3-benzyl-5-(2-hydroxyethyl)-4-methyl thiazolium bromide in 1 mL of distilled water was then added, the resulting solution was homogenized, immediately frozen at −50° C. and lyophilized. The amount of the active iodine was determined by reductive titration with sodium thiosulphate to be 3.5%.

### Example 24

Preparation of Hyaluronan-Oxythiamine-I₃ (HA-OB1-I₃) Lyophilizate

4.0 mg of KI and 2.7 mg of I₂ were added to a solution of hyaluronan (0.4 g, Mw 500 kg.mol⁻¹) in 100 mL of distilled water and 1 mL of acetic acid, and the resulting mixture was stirred for 24 hours at laboratory temperature. A solution of 45 mg of oxythiamine hydrochloride in 1 mL of distilled water was then added, the resulting solution was homogenized, immediately frozen at −50° C. and lyophilized. The amount of the active iodine was determined by reductive titration with sodium thiosulphate to be 0.5%.

### Example 25

Preparation of a Non-Woven Fabric from Staple Fibers Hyaluronan-Thiamine-I₃ (HA-B1-I₃)—Coating

1% aqueous HA solution was extruded through a nozzle with an inner diameter of 0.6 mm into a non-stationary coagulation bath consisting of 100% isopropanol at room temperature, which circumfluents the nozzle at 3 m.s⁻¹. The solution is precipitated into 3-4 cm long fibers. The crude fibers are shortened in a blender for 30 seconds at a ratio of 1 g of fibers per 1 liter of coagulation bath. The resulting fibrous dispersion having a fiber length of 3-4 mm is filtered through a substrate consisting of PAD knitted fabric and dried on a drying plate allowing fixation of the shape of the resulting fabric during drying. The resulting layer was separated from the substrate as a self-supporting layer. The fabric so formed was formatted to the desired size and immersed in a solution of NaI₃+B1 in ethanol/water 9/1 (Example 10). The fabric was placed on a shaker and subjected to NaI₃+B1 solution for 60 minutes at 20° C. and shaking speed of 80 oscillations per minute. The treated fabric is dried at laboratory temperature. The amount of the active iodine was determined by reductive titration with sodium thiosulphate to be 1.8%.

### Example 26

Preparation of a Non-Woven Fabric from Staple Fibers from Palmitoyl Hyaluronan-Thiamin-I₃ (HA-B1-I₃)—Coating

1% palmitoyl HA solution (prepared as described in Example 4), dissolved in a mixture of water and isopropanol in volume ratio 1:1, was extruded through a nozzle with an inner diameter of 0.6 mm into a non-stationary coagulation bath consisting of 90% isopropanol at room temperature, which circumfluents the nozzle at 3 m.s⁻¹. The solution is precipitated into 3-4 cm long fibers. The crude fibers are dehydrated in 100% acetone and shortened in a blender for 10 seconds at a ratio of 0.9 g of fibers per 1 liter of 100% isopropanol. The resulting fibrous dispersion having a fiber length of 3-4 mm is filtered through a substrate consisting of PAD knitted fabric and dried at 40° C. on a drying plate allowing fixation of the shape of the resulting fabric during drying. The resulting layer was separated from the substrate as a self-supporting layer. The fabric so formed was formatted to the desired size and immersed in a solution of NaI₃+B1 in ethanol/water 9/1 (Example 10). The fabric was placed on a shaker and exposed to NaI₃+B1 solution for 70 minutes at 20° C. and shaking speed of 80 oscillations per minute.

The treated fabric is dried at laboratory temperature. The amount of the active iodine was determined by reductive titration with sodium thiosulphate to be 1.5%.

### Example 27

Preparation of the Nanofiber Layer of Hyaluronan-Thiamin-I₃ (HA-B1-I₃)—Coating

An aqueous solution of the following composition was prepared to prepare a nanofibre layer containing hyaluronic acid. The concentration of HA having the molecular weight of 82 kg.mol⁻¹ in the dry matter was 80%, the concentration of polyethylene oxide with the molecular weight of 400 kg. mol⁻¹ was 5%, the concentration of polyvinyl alcohol with a molecular weight of 200 kg.mol⁻¹ was 15%, the concentration of the total dry matter was 6%. The solution was filled into a syringe and electrostatically spun onto a plate collector using a needle-free linear nozzle, voltage of 45 kV and distance of 18 cm between the emitter and the collector. The fibers have the dimension of 110±27 nm. This material was completely immersed in a solution of NaI₃+B1 in ethanol/water 6/1 (Example 9) for 48 hours at 20° C. Then the material was collected and immersed in isopropanol for 2 seconds, collected and dried at laboratory temperature. The amount of the active iodine was determined by reductive titration with sodium thiosulphate to be 8%.

### Example 28

Preparation of a Self-Supporting Film from Hyaluronan-Thiamine-I₃ (HA-B1-I₃)—Coating

Preparation of the film was carried out in a specialized drying apparatus where the film was dried in closed space. The device is equipped with a bottom and top plate with adjustable temperature. The device is further described in (Foglarova et al., CZ Application No. PV2015-166, Foglarova M. et al., Carbohydrate Polymers 2016, 144, 68-75). 240 mg of sodium hyaluronate having the molecular weight of 330 kg.mol⁻¹ was dissolved in 24 mL, of demineralized water and the mixture was stirred for at least 18 hours. The solution was then charged on a pad of the drying apparatus (hydrophobized glass) and dried in closed space at the bottom plate temperature of 50° C. and the top plate temperature of 20° C. The drying time was 20 hours. After drying, the film was removed from the pad and stored for further use. This material was then completely immersed in a solution of NaI₃+B1 in ethanol/water 6/1 (Example 9) for 72 hours at 20° C. Then the material was collected and immersed in isopropanol for 2 seconds, collected and dried at laboratory temperature. The amount of the active iodine was determined by reductive titration with sodium thiosulphate to be 0.1%.

### Example 29

Preparation of a Self-Supporting Film from Palmitoyl Hyaluronan-Thiamine-I₃ (palmHA-B1-I₃)—Coating

The film preparation device is described in the Example 28. 240 mg of palmitoyl derivative of sodium hyaluronan, described in Example 4, was dissolved in 24 mL of an aqueous solution of 2-propanol (50% w/w) and the mixture was stirred for at least 18 hours. The solution was then dispensed on a pad of the drying apparatus (hydrophobized glass) and dried in closed space at the bottom plate temperature of 50° C. and the top plate temperature of 40° C. The drying time was 20 hours. After drying, the film was removed from the pad and stored for further use. This material was then completely immersed in a solution of NaI₃+B1 in ethanol/water 6/1 (Example 9) for 72 hours at 20° C. Then the material was collected and immersed in isopropanol for 2 seconds, collected and dried at laboratory temperature. The amount of the active iodine was determined by reductive titration with sodium thiosulphate to be be 0.2%.

### Example 30

Preparation of a Self-Supporting Film from Lauroyl Hyaluronan-Thiamine-I₃ (laurHA-B1-I₃)—Coating

The film preparation device is described in the Example 28.240 mg of lauroyl derivative of sodium hyaluronan, described in Example 3, was dissolved in 24 mL of an aqueous solution of 2-propanol (50% w/w) and the mixture was stirred for at least 18 hours. The solution was then charged on a pad, of the drying apparatus (hydrophobized glass) and dried in closed space at the bottom plate temperature of 50° C. and the top plate temperature of 40° C. The drying time was 20 hours. After drying, the film was removed from the pad and stored for further use. This material was then completely immersed in a solution of NaI₃+B1 in ethanol/water 6/1 (Example 9) for 24 hours at 20° C. Then the material was collected and immersed in isopropanol for 2 seconds, collected and dried at laboratory temperature. The amount of the active iodine was determined by reductive titration with sodium thiosulphate to be 0.3%.

### Example 31

Preparation of the Hyaluronan-Thiamine-I₃ (HA-B1-I₃) Staple Fiber Layer—Coating

The non-woven fabric was produced by combining staple microfibers that are prepared by the wet spinning method in a non-stationary coagulation bath. Hyaluronic acid of the molecular weight 1,000 kg.mol⁻¹ was used. The coagulation bath consists of isopropanol. The precipitated fibers were then shortened by grinding, filtered to a substrate, dried and compressed. The resulting layer was separated from the substrate as a self-supporting layer, This material was then completely immersed in a solution of NaI₃B 1 in ethanol/water 9/1 (Example 10) for 1 hour at 20° C. Then it was dried at laboratory temperature. The amount of the active iodine was determined by reductive titration with sodium thiosulphate to be 1.8%.

### Example 32

Preparation of Knitted Fabric from Hyaluronan-Thiamine-I₃ (HA-B1-I₃) Fibers—Coating

An endless fiber of hyaluronan having the molecular weight of 600 kDa was used to produce the knitted fabric; the fiber fineness was 10 tex, the strength 1.1 N and the ductility 9.8%. Three fibers were pooled and twisted on a ring machine at feeding 10 m/min and spindle speeds of 3,000 min⁻¹; the resulting twist had the value of 300 m⁻¹. A two-sided tricot knitted fabric with closed stitches was knitted from threads on a double bed warp knitting machine. The knitted fabric was then washed in ethanol at 40° C. for 20 minutes. The resulting knitted fabric strip was 11 mm wide, had a mas per unit area of 99 g.m⁻² and stitches density 36 cm⁻². This material was then completely immersed in a solution of KI₃+B1 in ethanol/water 6/1 (Example 6) for 24 hours at 20° C. Then the material was collected and immersed in isopropanol for 2 seconds, collected and dried at laboratory temperature. The amount of the active iodine was determined by reductive titration with sodium thiosulphate to be 0.1%.

### Example 33

Preparation of Knitted Fabric from Palmitoyl Hyaluronan-Thiamine-I₃ (palmHA-B1-I₃) Fibers—Coating

An endless fiber of palmitoyl hyaluronan having the molecular weight of 320 kDa and the degree of substitution 30% (determined by NMR) was used to produce a knitted fabric; the fiber fineness was 9 tex, the strength of 0.6 N and the ductility of 21%. Three fibers were pooled and twisted on a ring machine at feeding 10 m/min and spindle speeds 3,000 min⁻¹; the resulting twist had the value 300 m⁻¹. A two-sided tricot knitted fabric with closed stitches was knitted from threads on a double bed warp knitting machine. The knitted fabric was then washed in ethanol at 40° C. for 20 minutes. The resulting knitted fabric strip was 11 mm wide, had a mas per unit area of 91 g.m⁻² and stitches density 36 cm². This material was then completely immersed in a solution of KI₃+B1 in ethanol/water 9/1 (Example 7) for 15 hours at 20° C.

Then the material was collected and immersed in isopropanol for 2 seconds, collected and dried at laboratory temperature. The amount of the active iodine was determined by reductive titration with sodium thiosulphate to be 0.3%.

### Example 34

Preparation of Alginate-Thiamine-I₃ Lyophilizate

40 mg of KI and 27 mg of I₂ were added to a solution of sodium alginate (0.4 g, Mw 400 kg.mol⁻¹) in 100 mL of distilled water and 1 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 4.4%.

### Example 35

Preparation of Oxycellulose-Thiamine-I₃ Lyophilizate

40 mg of KI and 27 mg of I₂ were added to a solution of oxycellulose (0.4 g, Mw 50 kg.mol⁻¹) in 100 mL of distilled water and 1 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 3.3%.

### Example 36

Preparation of Hydroxyethyl Cellulose-Thiamine-I₃ Lyophilizate

40 mg of KI and 27 mg of I₂ were added to a solution of hydroxyethyl cellulose (0.4 g, Mw 720 kg.mol⁻¹) in 100 mL of distilled water and 1 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 6.1%.

### Example 37

Preparation of Carboxymethyl Cellulose-Thiamine-I₃ Lyophilizate

40 mg of KI and 27 mg of I₂ were added to a solution of carboxymethyl cellulose (0.4 g, Mw 250 kg.mol⁻¹) in 100 mL of distilled water and 1 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50 ° C. and lyophilized. The amount of the active iodine was determined by reductive titration with sodium thiosulphate to be 4.6%.

### Example 38

Preparation of Oxycellulose/Hyaluronan-Thiamine-I₃ Lyophilizate

40 mg of KI and 27 mg of 12 were added to a solution of oxycellulose (0.3 g, Mw 50 kg.mol⁻¹) and hyaluronic acid (0.1 g, Mw 500 kg.mol⁻¹) in 100 mL of distilled water and 1 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 4%.

### Example 39

Preparation of Alginate/Hyaluronan-Thiamine-I₃ Lyophilizate

40 mg of KI and 27 mg of I₂ were added to a solution of sodium alginate (0.3 g, Mw 400 kg.mol⁻¹) and hyaluronic acid (0.1 g, Mw 500 kg.mol⁻¹) in 100 mL of distilled water and 1 mL of acetic acid, and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 4.3%.

### Example 40

Preparation of Carboxymethyl Cellulose/Hyaluronan-Thiamine-I₃ Lyophilizate

40 mg of KI and 27 mg of I₂ were added to a solution of carboxymethyl cellulose (0.3 g, Mw 250 kg.mol⁻¹) and hyaluronic acid (0.1 g, Mw 500 kg.mol⁻¹) in 100 mL of distilled water and 1 mL of acetic acid and the resulting mixture was stirred for 24 hours at laboratory temperature. A solution of 38 mg of thiamine hydrochloride in 1 mL of distilled water was then added, the resulting solution was homogenized, immediately frozen at −50° C. and lyophilized. The amount of the active iodine was determined by reductive titration with sodium thiosulphate to be 4.2%.

### Example 41

In Vitro Antimicrobial Activity Assay (FIGS. 1 and 2):

Suspensions of individual tested microorganisms were prepared at an approximate concentration of 10⁵ CFU/mL. Onto the surface of tryptone soya agar in Petri dishes, 100 μL of suspension (approximately 10⁴ CFU of microorganisms on the dish) was applied. The suspension was evenly spread over the entire surface of the dish with a sterile loop. After the suspension was absorbed by agar, the tested samples were transferred in a sterile way onto the surface of the agar in the form of squares. The dishes with bacterial test strains were cultured at 37° C. for 24 hours. Lyophilizates with the antimicrobial substance HA-B1-I₃, HA-TH-I₃ and HA-BTH-I₃ (prepared according to the Examples 13, 22, 23) were tested, where analogous lyophilizates without the active substance HA-TH, HA-BTH and lyophilizates with HA alone were used as controls. Squares of the weight of 15-20 mg and approximate dimensions of 15×15×2 mm were prepared, with 0.7-1.3 mg of potassium triiodide or without potassium triiodide. For efficiency testing, a diffusion plate method (2D layout) was chosen. A non-selective soil (tryptone soya agar) was used for cultivation. The square samples were tested on 2 microorganisms—Escherichia coli (G-rod) and Staphylococcus aureus (G+coccus). Figures 1 and 2 clearly show a considerably higher efficiency of lyophilizates of the invention in comparison with lyophilizates without triiodide or with HA itself.

### Example 42

Testing of the Tolerance and the Effect on Wound Healing (FIG. 3).

A one-week analysis was conducted to compare the effect of HA-B1-I₃ lyophilizate (prepared according to the Example 13) on the course of the wound healing. The study was focused primarily on the tolerance of the preparation and the comparison of its efficacy with the standard wound healing agent with a proven effect, which is a dressing containing an active-layer, which is a combination of hyaluronan and the antimicrobial substance octenidine (HA-octenidine). For testing, a bandage of the same composition as HA-octenidine dressing was used, but the active layer was replaced with the HA-B1-I₃ lyophilizate. The study was conducted in a patient where half of the wound was always treated with a HA-B1-I₃ lyophilizate bandage (indicated as HyB₁ in FIG. 3), the second half with a standard HA-octenidine dressing.

In the patient, the bandage was tolerated without any negative subjective or objective problems. The wound healing course during the observed one-week period was comparable to the healing when HA-octenidine preparation was used. On the wounds covered by HA-octenidine and HA-B1-I₃ lyophilizate no signs of infectious or inflammatory complications were recorded. Thus, it can be concluded that the effect of the new HA-B₁-I₃ complex is comparable to that of the HA-octenidine standard dressing. The preparation according to the invention is advantageous in comparison with the octenidine preparation especially because iodine is considerably more biocompatible compared to octenidine, and therefore much more suitable, for example, for implantable materials.

The terms “comprising” or “comprise” are used herein in their broadest sense to mean and encompass the notions of “including,” “include,” “consist(ing) essentially of,” and “consist(ing) of. The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-25, ±0-10, ±0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1.-14. (canceled)

15. An antimicrobial composition, comprising:
a polysaccharide component comprising a polysaccharide selected from the group of hyaluronic acid, sodium alginate, oxycellulose, carboxymethyl cellulose, and hydroxyethyl cellulose or a chemically modified hyaluronic acid derivative comprising at least one of a substituted —OH group of formula —O—C(O)—R² and a substituted —C(O)—OH group of formula —C(O)—OR² with each R² independently being an a linear or aromatic group having 1-15 carbon atoms, the polysaccharide or chemically modified hyaluronic acid derivative having a molecular weight in a range from 5×10³ to 1×10⁶ g.mol⁻¹;
a thiazole-based stabilizer selected from the group of thiamine, oxythiamine hydrochloride, 5-(2-hydroxyethyl)-3,4-dimethyl thiazolium iodide, 3-benzyl-5-(2-hydroxyethyl)-4-methyl thiazolium bromide, and combinations thereof; and
sodium or potassium triiodide.

16. The composition of claim 15, wherein the composition is in a solid form selected from the group of lyophilizate, self-supporting film, non-woven fabric, endless fiber, woven fabric, knitted fabric, plaited fabric, a nanofiber layer, and combinations thereof.

17. A method of preparing the composition of claim 15, the method comprising:
combining the thiazole-based stabilizer, the sodium or potassium triiodide, and the polysaccharide component to form the composition.

18. The method of claim 17, comprising:
combining the thiazole-based stabilizer and the sodium or potassium triiodide in a molar ratio of the thiazole-based stabilizer to the sodium or potassium triiodide in a range from 1:1 to 5:1 to form a stabilizer/triiodide mixture; and
combining the stabilizer/triiodide mixture and the polysaccharide component to form the composition;
wherein the sodium or potassium triiodide is utilized in an amount of from 0.2 to 10% by weight, based on a total weight of the polysaccharide component.

19. The method claim 18, further comprising:
combining the polysaccharide or chemically modified hyaluronic acid derivative and a water/acetic acid solvent mixture comprising water and acetic acid in a volume ratio of from 20:1 to 200:1, thereby give the polysaccharide component as a polysaccharide/solvent mixture, where the polysaccharide or chemically modified hyaluronic acid derivative is present in an amount of from 0.2 to 6 wt. % based on a total weight of the polysaccharide/solvent mixture;
wherein combining the stabilizer/triiodide mixture and the polysaccharide component is further defined as combining the stabilizer/triiodide mixture and the polysaccharide/solvent mixture to form the composition.

20. The method of claim 19, wherein the water/acetic acid solvent mixture comprises a volume ratio of water to acetic acid solvent of 100:1.

21. The method of claim 18, further comprising:
combining the stabilizer/triiodide mixture and an ethanol/water solvent mixture to form a stabilizer/triiodide/solvent solution;
wherein combining the stabilizer/triiodide mixture and the polysaccharide component is further defined as applying the stabilizer/triiodide/solvent solution to the polysaccharide or chemically modified derivative thereof to form the composition.

22. The method of claim 21, wherein the stabilizer/triiodide/solvent solution is applied to the polysaccharide component over an application time of from 10 minutes to 72 hours and at a temperature of from 5 to 40° C.

23. The method of claim 21, wherein applying the stabilizer/triiodide/solvent solution to the polysaccharide component comprises spraying the stabilizer/triiodide/solvent solution onto the polysaccharide or chemically modified hyaluronic acid derivative or immersing the polysaccharide or chemically modified hyaluronic acid derivative in the stabilizer/triiodide/solvent solution for a time period of from 5 to 15 hours.

24. The method of claim 21, wherein the sodium or potassium triiodide is present in the stabilizer/triiodide/solvent solution in an amount of from 0.2 to 10% by weight based on a total weight of the stabilizer/triiodide/solvent solution, wherein the thiazole-based stabilizer and the sodium or potassium triiodide are combined in a molar ratio of from 1:1 to 5:1, and wherein the volume ratio of ethanol to water of the ethanol/water solvent mixture is from 3:1 to 9:1.

25. The method of claim 24, wherein the thiazole-based stabilizer and the sodium or potassium triiodide are combined in a molar ratio of from 1.1:1.

26. A medical device comprising the composition prepared according to claim

17.

27. The medical device of claim 26, wherein the composition is in a final form selected from the group of lyophilizate, self-supporting film, nanofibre layer, non-woven fabric, fiber, knitted fabric, woven fabric, plaited fabric, and combinations thereof.

28. The medical device of claim 26, wherein the medical device is in a form of a wound dressing or an implantable medical device.

29. A method of utilizing the composition of claim 15 for preparing a wound dressings, the method comprising:
providing the composition; and
preparing the wound dressing utilizing the composition.

30. A method of utilizing the composition of claim 15 for preparing an implantable medical device, the method comprising:
providing the composition; and
preparing the implantable medical device utilizing the composition.
